(19) United States
(12) Patent Application Publication
(10) Pub. No.: US 2006/0199207 A1
(43) Pub. Date: Sep. 7, 2006

(54) Self-assembly of molecules using combinatorial hybridization

(76) Inventor: Stefan M. Matysiak, Montara, CA (US)

Correspondence Address: JONES DAY, 51 Louisiana Avenue N.W., Washington, DC, 20001-2113, US

(21) Appl. No.: 11/360,576

(22) Filed: Feb. 24, 2006

Related U.S. Application Data
(60) Provisional application No. 60/655,960, filed on Feb. 24, 2005.

(51) Int. Cl. C40B 40/08; C12Q 1/68; C12M 1/34; C07K 9/00

(52) U.S. Cl. 435/6; 530/322; 435/287.2

(57) ABSTRACT

Simple and convenient methods for arranging molecules of interest in a pre-determined pattern are described. The methods use combinatorial hybridization based on interactions between complementary nucleic acid sequences to arrange the molecules of interest. The resulting arrangements, kits containing the components used in the methods, and methods of using the resulting arrangements are also disclosed.

This application claims priority to U.S. Provisional Application No. 60/655,960, filed Feb. 24, 2005, the entirety of which is incorporated herein by reference.

### 1. FIELD OF THE INVENTION

This invention relates to ordered arrangements of molecules and methods of making them using combinatorial hybridization. Methods of using the arrangements are also encompassed by the invention.

### 2. BACKGROUND OF THE INVENTION

Ordered arrangements of biomolecules and small molecules are useful in a wide variety of applications. One example is the use of nucleic acid arrays for the profiling of gene expression. For example, profiling of gene expression using mRNA monitoring can be used to study the internal life of cells.

Gene expression profiling has a wide variety of applications. For example, it can be used to identify protein targets for therapeutics and to monitor the influence of therapeutics in vivo, and thus to devise “point of care” diagnostics.

Unfortunately, there are several obstacles that can hamper the reliability of gene expression profiling. First, mRNA levels do not always correlate with protein levels (e.g., with a correlation factor >0.5). In addition, one mRNA does not necessarily code for one protein, mainly due to alternative splicing between exons. Furthermore, mRNAs cannot provide precise information concerning the resulting proteins, because: 1) the functions of proteins are affected by factors such as post-translational modification; 2) proteins have varying half-lives; 3) proteins can be compartmentalized into different cellular locations in ways that can affect their activities; and 4) some proteins are functionally defunct until they are assembled into large complexes. “The Current state of Proteomic Technology,” www.chiresource.com/newsarticles/issue3_{—}1.ASP.

To address these problems, various attempts to make and use protein chips that allow the direct determination of the expressions and/or functions of proteins have been reported. See, e.g., Paul Cutler, Review: “Protein arrays: The current state-of-the-art,” Proteomics, 3: 3-18 (2003). However, the manufacture of protein chips has proven to be more difficult than that of nucleic acid arrays. Because proteins can easily unfold when coming in contact with inappropriate surface or environment, they require more delicate handling than DNA. Furthermore, the detection of nucleic acids based on complementarity of sequences is much easier than the detection of proteins using techniques such as mass spectrometric analysis and interaction with certain molecules that specifically recognize their molecular structure. Therefore, a need exists for simple and reliable methods to assess the expression and function of proteins.

Simple and reliable methods of arranging molecules of interest in an ordered fashion would also provide a valuable tool for drug discovery, biomolecule assays, and characterization of the mechanisms of action of biomolecules.

### 3. SUMMARY OF THE INVENTION

This invention is directed, in part, to a new approach of organizing molecules of interest using combinatorial hybridization and three-dimensional self assembling molecular systems. These systems use a plurality of anchors comprising one or more nucleic acid fragments immobilized on a surface. Conjugates of nucleic acid fragments and the molecules to be organized are then hybridized. Hybridization occurs because each of the conjugates' nucleic acid fragments has a sequence complementary to one of the nucleic acid fragments present in the anchors. The result is an ordered array of the molecules of interest.

These systems can be used to organize and analyze molecules such as, but not limited to: peptides, including those comprising L- or D-amino acids; peptoids; proteins; steroids or analogues thereof; hormones; carbohydrates; polycarbohydrates; aminoglycosides; aptamers of L or D oligoribonucleotides; nucleoside antibiotics, including L-nucleoside analogues; oligoglycosids; polyketid antibiotics such as macrolids, polyenes, oligolactones, polyethers, tetracycline, and anthracycline; p-chinoid macrolactams; terpenoids such as isopren and analogues thereof; peptide antibiotics; and benzodiazepine.

Accordingly, this invention encompasses an array comprising a plurality of conjugates and a plurality of anchors, wherein:
each of the conjugates comprises a molecule bound to a nucleic acid fragment;
each of the anchors is immobilized on a surface and comprises at least two nucleic acid fragments; and
the nucleic acid fragment of each conjugate is hybridized to a nucleic acid fragment of one of the anchors.
   Preferably, the molecule of each conjugate is not the same as the molecule of any of the other conjugates.

In one embodiment, the molecule and nucleic acid fragment forming a conjugate are covalently bound.

This invention also encompasses a method of arranging molecules comprising:
(a) immobilizing a first set of nucleic acid fragments with known sequences in a predetermined pattern on a surface to form anchors;
(b) contacting the anchors with a mixture comprising conjugates of a second set of nucleic acid fragments and the molecules to be arranged, wherein the nucleic acid fragment in each conjugate has a sequence complementary to at least part of one of the nucleic acid fragments in the anchors; and
(c) incubating the anchors and the mixture for a time and under conditions sufficient for the conjugates to bind to the anchors, thereby arranging the molecules.
   Thus, the bound conjugates provide an array of the molecules arranged according to the pattern of immobilization of the first set of nucleic acid fragments.

These ordered arrays of the molecules of interest (e.g., peptides) can be used in a wide variety of applications. One such application is obtaining “fingerprints” of proteins. Thus, this invention also encompasses a method of characterizing a protein comprising:
(a) immobilizing a first set of nucleic acid fragments with known sequences in a predetermined pattern on a surface to form anchors;
(b) contacting the anchors with a mixture comprising conjugates of a second set of nucleic acid fragments and peptides, wherein the nucleic acid fragment in each conjugate has a sequence complementary to at least part of one of the nucleic acid fragments in the anchors;
(c) incubating the anchors and the mixture for a time and under conditions sufficient for the conjugates to bind to the anchors to provide an array of anchor-conjugate complexes;
(d) contacting the array with the protein for a time and under conditions sufficient for the protein to bind to one or more of the complexes; and
(e) detecting the binding of the protein to the complexes to obtain a binding pattern;
   wherein the binding pattern is characteristic of the protein.

Kits for protein and other assays based on methods of this invention, as well as hardware and software for computer-assisted automation of those methods, are also encompassed by this invention.

### 4. BRIEF DESCRIPTION OF FIGURES

Aspects of certain embodiments of the invention can be understood with reference to the attached figures.

FIG. 1 illustrates components used in self-assembly methods of the invention.

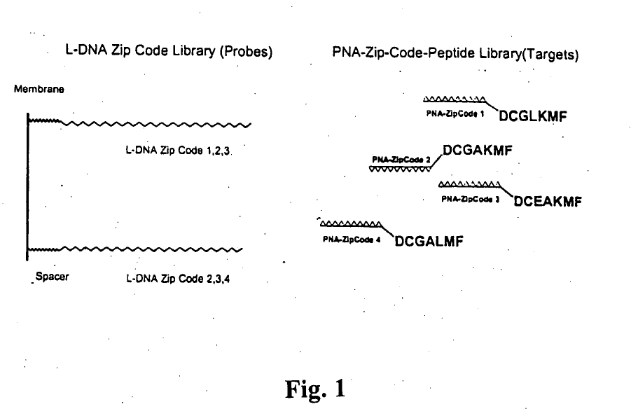

FIG. 2 illustrates an arrangement of peptide fragments, self-assembled according to methods of this invention.

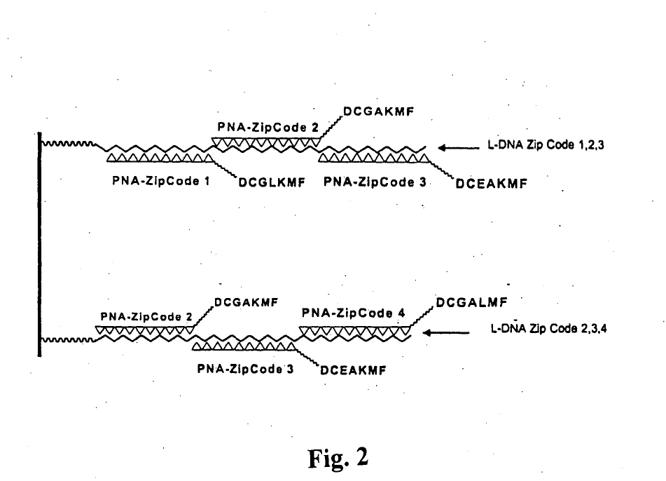

FIG. 3 illustrates the transmembrane structure of the G-protein coupled receptor (“GPCR”) Ste2p.

### 5. DETAILED DESCRIPTION OF THE INVENTION

This invention is directed, in part, to methods of arranging molecules of interest using self-assembly. This invention is also directed to the use and applications of such arrangements, and combinations, kits, and systems for preparing them. Methods of this invention utilize: a plurality of anchors, which comprise one or more nucleic acid fragments, and are immobilized on a surface or a support; a plurality of conjugates, each of which comprises a nucleic acid fragment having a specific affinity to at least a part of the anchors and conjugated to a molecule of interest. Preferably, the anchors are immobilized on the surface according to a predetermined pattern. The interaction between anchors and the conjugates provide a spontaneous “self-assembly” of the molecules of interest according to the pattern of immobilization of anchors on the surface.

In particular embodiments, the anchors and conjugates comprise nucleic acid fragments whose sequences are complementary to each other, so that the molecules of interest are arranged according to the interaction between the anchors and conjugates. As used herein, and unless otherwise specified, the term “complementary” means that a sequence is able to bind to a target sequence. The binding may result from interactions such as, but not limited to, nucleotide base parings (e.g., A-T/G-C).

In particular embodiments of the invention, a sequence is complementary when it hybridizes to its target sequence under high stringency conditions, i.e., conditions for hybridization and washing under which nucleic acid sequences, which are at least 60 percent (preferably greater than about 70, 80, or 90 percent) identical to each other, typically remain hybridized to each other. Such stringent conditions are known to those skilled in the art, and can be found, for example, in Current Protocols in Molecular Biology, John Wiley & Sons, N.Y. (1989), 6.3.1-6.3.6, which is incorporated herein by reference.

Examples of highly stringent hybridization conditions include, but not limited to: hybridization of the nucleotide sequences in 6x sodium chloride/sodium citrate (SSC) at about 45° C., followed by 0.2×SSC, 0.1% SDS at 50-65° C.; hybridization in 6× sodium chloride/sodium citrate (SSC) at about 45° C., followed by one or more washes in 0.2×SSC, 0.1% SDS at 50° C.; hybridization in 6× sodium chloride/sodium citrate (SSC) at about 45° C., followed by one or more washes in 0.2×SSC, 0.1% SDS at 55° C.; hybridization in 6× sodium chloride/sodium citrate (SSC) at about 45° C., followed by one or more washes in 0.2×SSC, 0.1% SDS at 60° C.; hybridization in 6× sodium chloride/sodium citrate (SSC) at about 45° C., followed by one or more washes in 0.2×SSC, 0.1% SDS at 65° C.; and hybridization in 0.5M sodium phosphate, 7% SDS at 65° C., followed by one or more washes at 0.2×SSC, 1% SDS at 65° C.

Depending on the conditions under which binding is sufficient to maintain the arrangement of the molecules of interest, a sequence complementary to a second sequence need not be 100 percent complementary to the second sequence. For example, a sequence can be complementary to a second sequence when at least about 70, 80, 90, or 95 percent of its nucleotides bind via matched base pairings with nucleotides of the second sequence.

One embodiment of this invention encompasses a method of arranging molecules of interest comprising:
(a) immobilizing a first set of nucleic acid fragments with known sequences in a predetermined pattern on a surface to form anchors;
(b) contacting the anchors with a mixture comprising conjugates of a second set of nucleic acid fragments and the molecules, wherein the nucleic acid fragment in each conjugate has a sequence complementary to at least part of one of the nucleic acid fragments in the anchors; and
(c) incubating the anchors and the mixture for a time and under conditions sufficient for the conjugates to bind to the anchors, thereby arranging the molecules. The resulting bound conjugates provide an array of the molecules arranged according to the pattern of immobilization of the first set of nucleic acid fragments.

Conjugates used in methods and compositions of the invention comprise at least one nucleic acid fragment attached to a molecule of interest. Preferably, the nucleic fragment is conjugated the molecule of interest with a sufficient K_{d} so that the conjugate does not fall apart upon its binding to an anchor. The nucleic acid fragment(s) and the molecule of interest can be covalently or non-covalently conjugated.

Another embodiment of this invention encompasses an array comprising a plurality of conjugates and a plurality of anchors, wherein:
each of the conjugates comprises a molecule bound to a nucleic acid fragment;
each of the anchors is immobilized on a surface and comprises at least two nucleic acid fragments; and
the nucleic acid fragment of each conjugate is hybridized to a nucleic acid fragment of one of the anchors.
   Preferably, the molecule of each conjugate is not the same as the molecule of any of the other conjugates.

As used herein, and unless otherwise specified, the term “array” means a spatial arrangement of molecules, which encompasses two- and three-dimensional arrangements. Certain array formats are referred to as a “chip” or “biochip.” See, e.g., Microarray Biochip Technology, M. Schena, Ed. (2000). An array may comprise a plurality of addressable locations configured so that each location is spatially addressable for high-throughput handling, robotic delivery, masking, or sampling of reagents, or for detection means including, but not limited to, scanning and light gathering.

Methods and compositions of this invention can be used in various applications. Examples of such applications include, but are not limited to: establishing binding “fingerprints” of known and unknown proteins; combining the “fingerprints” in an analytical chip for the determination of proteins in cell lysates; and monitoring the up- and down-regulation of protein levels in cells during, for example, medical treatments (point of care diagnostics) and development of therapeutic agents (target validation), and for identification of regulation mechanisms of enzymes.

In one embodiment, each of the anchors contains two or more nucleic acid fragments, each fragment is capable of binding to a conjugate. In another embodiment, at least two of the anchors comprise the same nucleic acid fragment, so that at least one nucleic acid fragment is present in two or more anchors.

Examples of molecules of interest that can be arranged using the invention include, but are not limited to: peptides, including those comprising L- or D-amino acids; peptoids; proteins; steroids or analogues thereof; hormones; carbohydrates; polycarbohydrates; aminoglycosides; aptamers of L or D oligoribonucleotides; nucleoside antibiotics, including L-nucleoside analogues; oligoglycosids; polyketid antibiotics such as macrolids, polyenes, oligolactones, polyethers, tetracycline, and anthracycline; p-chinoid macrolactams; terpenoids such as isopren and analogues thereof; peptide antibiotics; benzodiazepine; and any other molecules that can be stably conjugated to nucleic acid fragments.

In one embodiment, the molecules of interest are peptides. As used herein, and unless otherwise specified, the term “peptide” means a chain of two or more amino acids bound to each other via peptide bonds. The amino acids can be substituted or unsubstituted, and may be synthetic or a part of naturally occurring protein. Peptides can comprise one or more “unnatural” amino acids, such as, but not limited to, peptoids and D-amino acids. In a specific embodiment, the peptide is a tri-peptide.

Another embodiment of this invention encompasses a kit for protein assay based on methods and arrays of this invention, and equipment and software associated with (e.g., that implement) the methods of this invention in an automated, high-throughput context.

5.1 Anchors

Anchors comprise one or more nucleic acid fragments, and are immobilized on a surface, preferably in a pre-determined order. Nucleic acid fragments include, but are not limited to, fragments of DNA, RNA, and analogues and derivatives thereof.

As used herein, and unless otherwise specified, the term “nucleic acid” encompasses single- and double-stranded polynucleotides such as, but not limited to, DNA including L-DNA, RNA, peptide nucleic acid (“PNA”; for detailed explanation, see, e.g., Uhlmann et al., “PNA: Synthetic Polyamide Nucleic Acids with Unusual Binding Properties”), phosphothioate DNA, and other analogues and derivatives thereof See, e.g., Wang et al., “Six-membered carbocyclic nucleosides,” Advances in Antiviral Drug Design, 4: 119-145 (2004); and Pitsch et al., “Pentopyranosyl oligonucleotide systems: 9. The β-D-ribopyranosyl-(4′,2′)-oligonucleotide system (“pyranosyl-RNA”): Synthesis and resume of base-pairing properties,” Helvetica Chimica Acta, 86(12): 4270-4363 (2003).

Nucleic acids may include naturally occurring bases, as well as unnatural (e.g., synthetic) bases. See Chap. VI. Nucleotidomimetic Foldamers in Hill et al., “A Field Guide to Foldamers,” Chem. Rev., 101: 3893-4011 (2001). Backbones may contain bonds such as, but not limited to, phosphodiester, phosphotriester, phosphoramidate, phosphothioate, thioester, and peptide bonds. Nucleic acids can be in α or β conformation.

In some embodiments, nucleic acid fragments that can be used for the anchor structures invention include, but are not limited to, DNAs, in particular, L-DNAs, RNAs, peptide nucleic acids (“PNAs”), phosphothioate DNAs, and other analogues and derivatives thereof. Nucleic acid fragments may contain various modifications and analogues of standard bases, sugars, and internucleotide linkages. Such modifications and analogues may be disposed at any location and at any appropriate frequency of occurrence in the nucleic acid fragments.

Examples of analogues of standard bases include, but are not limited to, 2,6-diaminopurine, hypoxanthine, pseudouridine, C-5-propyne, isocytosine, and 2-thiopyrimidine.

Sugar modifications at the 2′ or 3′ position include, but are not limited to, C₁-C₆ alkoxy, C₁-C₆ alkyl, C₅-C₁₅ aryloxy, C₅-C₁₄ aryl, amino, C₁-C₆ alkylamino, fluoro, chloro, and bromo. Other sugar modifications include, but are not limited to, a 4′-α-anomeric nucleotide, a 1′-α-anomeric nucleotide, a 2′-4′ L-form LNA, a 2′-4′ D-form LNA, a 3′-4′ L-form LNA, and 3′-4′ D-form LNA.

In addition to the naturally occurring phosphodiester linkages, nucleic acid fragments may contain one or more internucleotide linkages comprising a phosphate analog such as, but not limited to, a phosphorothioate, a phosphorodithioate, a phosphotriester, and a phosphoramidate. Other linkages include, but are not limited to, those where the sugar/phosphate backbone of DNA or RNA has been replaced with one or more acyclic, achiral, and/or neutral polyamide linkages.

In one embodiment, the nucleic acid fragment is L-DNA. As used herein, and unless otherwise specified, the term “L-DNA” refers to nucleic acids comprising nucleotides in the “L” configuration. L-DNAs may contain modified nucleotides such as, but not limited to, those comprising ribose, arabinose, xylose, pyranose, 2′-deoxyribose, 2′,3′-dideoxyribose, 2′-fluororibose, 2′-chlororibose, 2′-O-methylribose, and 2′-deoxy-L-erythro-pentose. See, e.g., WO 03/059929 and EP 0540742 A1. L-DNAs also encompass heteroconfigurational oligonucleotides, such as those described in WO 03/059929. As used herein, and unless otherwise indicated, the term “heteroconfigurational oligonucleotide” refers to an oligonucleotide comprising nucleotides of different configurations, e.g., one or more portions of L-form nucleotides and one or more portions of D-form nucleotides. L-DNAs may be in α or β anomeric configurations.

In another embodiment, the nucleic acid fragment used in methods of this invention is PNA. PNA is one class of nucleic acids with modified internucleotide linkages. One example is the 2-aminoethylglycine polyamide linkage with bases attached to the linkage through amide bonds. See, e.g., WO 92/20702; Nielson, Science, 254: 1497-1500 (1991); Egholm, Nature, 365: 566-8 (1993). PNA can hybridize to its target compliment in either a parallel or anti-parallel orientation. However, the anti-parallel duplex (where the carboxy terminus of PNA is aligned with the 5′ terminus of DNA, and the amino terminus of PNA is aligned with the 3′ terminus of DNA) is typically more stable. Egholm, supra. PNA probes are known to bind target DNA sequences with high specificity and affinity. See, e.g., U.S. Pat. No. 6,110,676. PNAs used in methods of this invention may include PNA-DNA chimera, with or without regions comprising L-form nucleotides. PNA-DNA chimera can be synthesized by covalently linking PNA monomers and phosphoramidite nucleosides in virtually any combination or sequence. These methods include those disclosed in Vinayak, Nucleosides & Nucleotides, 16: 1653-56 (1997); Uhlmann, Angew. Chem., Intl. Ed. Eng., 35: 2632-5 (1996); EP 829542; Van der Laan, Tetrahedron Lett., 38: 2249-52 (1997); and Van der Laan, Bioorg. Med. Chem. Lett., 8: 663-8 (1998). All of the above-cited references are incorporated herein in their entireties.

In one embodiment, at least one of the nucleic acid fragments in each anchor structure is L-DNA. In another embodiment, at least one of the nucleic acid fragments is PNA.

In this invention, anchors are formed by immobilizing nucleic acid fragments on a surface. Any solid phase material upon which a nucleic acid fragment can be attached or immobilized may be used as a surface. Thus, the term “surface” encompasses “solid support,” “support,” “resin,” and “solid phase.” Surfaces can exist in a wide variety of structures and geometries, such as, but not limited to, beads, pellets, disks, capillaries, hollow fibers, needles, solid fibers, wells, depressions, random shapes, thin films, membranes, and any solid surface with addressable loci. Surfaces can be porous or non-porous. Surfaces can be planar or non-planar. In some embodiments, where a non-planar surface (e.g., a well or capillary) is used, the nucleic acid fragments can be arranged so that the resulting self-assembled arrangement provides a three dimensional binding structure, which can be advantageously used for applications such as, but not limited to, the determination of protein/enzyme binding pocket structures.

Surfaces can be made from a variety of materials. Examples include, but are not limited to:
1) glass, silica, or gallium wafers;
2) electroconductive surface like metals such as, but not limited to, alumina, platinum, gold, nickel, copper, zinc, tin, palladium, and silver, and oxides of metals or metalloids;
3) transparent electroconductive surfaces such as, but not limited to, indiumtinoxide (ITO)
4) semiconductors such as, but not limited to, lithium niobate, gallium arsenide, and indium phosphide;
5) non electroconductive organic polymers such as, but not limited to: agarose and other polysaccharides; collagen; cellulose and derivatives thereof; acrylamides; dextran derivatives and co-polymers; nylon and co-polymers; agarose-polyacrylamide blends; methacrylate derivatives and co-polymers; polycarbonate; polyvinylchloride; PTFE; PTE; polystyrene and its co-polymers; polyvinyl alcohols; polyethylene-co-acrylic acid; polyethylene-co-methacrylic acid; polyethylene-co-ethylacrylate; polyethylene-co-methyl acrylate; polypropylene-co-acrylic acid; polypropylene-co-methyl-acrylic acid; polypropylene-co-ethylacrylate; polypropylene-co-methyl acrylate; polyethylene-co-vinyl acetate; polypropylene-co-vinyl acetate; polyethylene-co-maleic anhydride; polypropylene-co-maleic anhydride; polyurethane based polymers; and electro-conductive derivatives of said organic polymers; and
6) liposomes and micelles.
   Additional materials are known by those skilled in the art. Surface materials can be commercially obtained or made using well-known methods.

In one embodiment, appropriate surface derivatization processes can be used to generate surfaces with patterns of hydrophilic areas within otherwise hydrophobic surroundings. These processes are well-known in the art. In general, and without being limited by a particular theory, surface tension can be used to facilitate the exact and efficient deposition of biopolymers in aqueous or non-aqueous solutions, depending on solvent used, and the covalent or non-covalent attachment thereafter.

In one specific embodiment, the surface can be a microscope slide patterned with through-going holes comprising hydrophilic surfaces. Stacked microscope slides can be filled with hydrophilic liquid using a capillary, by putting the capillary through a specific hole of the stacked microscope slide. Without being limited by a particular theory, capillary forces and external air pressure allow the filling of the holes with substantially the same volume of liquid. This process can be used for, for example: immobilizing the anchor structures; adding the conjugates; and adding the sample liquid.

Nucleic acid fragments can be immobilized on surfaces using any of a variety of methods known in the art. Examples include, but are not limited to, absorption, adsorption, and covalent binding to the support, either directly or indirectly through a linker structure. Examples of linker structures include, but are not limited to, disulfide linkages, thioester bonds, hindered disulfide bonds, and covalent bonds between free reactive groups, such as amine and thiol groups and other groups known in the art. See, e.g., Pierce,

ImmunoTechnology Catalogue & Handbook.

Generally, to effect immobilization, a solution of nucleic acid fragments, with or without linker structures, is contacted with a surface material. Various methods are known for attaching nucleic acid fragments to a support. See, e.g., U.S. Pat. No. 6,023,540. For example, nucleic acid fragments can be attached to a support using photochemically active reagents, such as psoralen compounds, and a coupling agent, which attaches the photoreagent to the substrate (see, e.g., U.S. Pat. Nos. 4,542,102 and 4,562,157). Other methods include, but are not limited to: oxime coupling; chemical conjugation (e.g., as described in Section 5.2 below); in situ synthesis techniques (see, e.g., U.S. Pat. No. 5,436,327); light-directed in situ synthesis techniques (see, e.g., U.S. Pat. No. 5,744,305); robotic spotting techniques (see, e.g., U.S. Pat. Nos. 5,807,522 and 5,631,134); attachment of oligonucleotides to arrays and beads according to the method described in U.S. Pat. No. 6,023,540; and immobilization of L-form oligonucleotides on silicon wafers according to the method described in U.S. Pat. No. 5,545,531. Other methods of immobilization that can be used in connection with methods of this invention include, but are not limited to, those described in WO 02/57422, Guillaumie el al., Bioconjugate Chemistry, 13(2): 285-294 (2002), and Chan et al., Langmuir, 18(2): 311-313 (2002). All of the above-cited references are incorporated herein by reference in their entireties.

In one specific embodiment, immobilization is achieved using chemical conjugation, by first activating a porous nylon membrane with Di-succinoylcarbonate (DSC), and covalently attach the DNA oligomer via a terminal primary amine function.

In another embodiment, a stable, but non covalent, attachment is achieved by using the hydrophobic interaction of the polyperfluoro-tagged biopolymer with a perfluorinated surfaces (see, e.g., Beller et al., Helvetica Chimica Acta, 88: 171 (2005)), or the host-guest interaction of an amino terminated biopolymer with a surface comprising calixcrown-5 derivatives (see, e.g., Lee et al., Proteomics, 3: 2289-2304(2003)).

Other immobilization methods include, but are not limited to: immobilization of DNA via oligonucleotides containing an aldehyde or carboxylic acid group at the 5′ terminus (see, e.g., Kremsky et al., Nucleic Acids Res., 15(7): 2891-2909 (1987)); and covalently attaching spacer molecules with a terminal electrophilic functional group (e.g., alkylhalogenides, activated esters, azlactones, expoxides, ketones, and aldehydes) to a surface, and attaching a biopolymer with a reactive nucleophilc group (e.g., thiols, amines, semicarbazides, hydrazines, and aminooxy). In a particular embodiment, the electophilic group is an aldehyde, and the nucleophilic group is an aminooxy.

5.2 Conjugates

Conjugates used in this invention comprise a nucleic acid fragment and a molecule to be arranged (also referred to herein as “molecule of interest”). The types of nucleic acid fragments that can be used for the conjugates are the same as those used for anchor structures described above.

The molecules to be arranged will depend on the application to which this invention is put. Examples of the molecules include, but not limited to: organic compounds; inorganic compounds; metal complexes; receptors; enzymes; antibodies; proteins; nucleic acids; peptide nucleic acids; oligosaccharides; lipids; lipoproteins; amino acids; peptides; peptidomimetics; carbohydrates; cofactors; drugs; prodrugs; lectins; sugars; glycoproteins; biomolecules; macromolecules; biopolymers; non-bio polymers; sub-cellular structures; viruses, or portions thereof such as viral vectors and viral capsids; phages, or portions thereof such as phage vectosr and phage capsids; cells, or portions thereof; and other biological or chemical materials that can be conjugated to the nucleic acid fragments used in the conjugates.

In specific embodiments of the invention, the molecules to be arranged are: peptides, including those comprising L- or D-amino acids; peptoids; proteins; steroids or analogues thereof; hormones; carbohydrates; polycarbohydrates; aminoglycosides; aptamers of L or D oligoribonucleotides; nucleoside antibiotics, including L-nucleoside analogues; oligoglycosids; polyketid antibiotics such as macrolids, polyenes, oligolactones, polyethers, tetracycline, and anthracycline; p-chinoid macrolactams; terpenoids such as isopren and analogues thereof; peptide antibiotics; and benzodiazepine. In one embodiment, the molecules to be arranged are peptides. In a specific embodiment, the molecules to be arranged are tri-peptides.

Molecules can be conjugated to the nucleic acids using any methods known in the art, as well as those described herein. See, e.g., Hermanson, Bioconjugate Chemistry (1996). Generally, molecules to be arranged can be conjugated to the nucleic acid fragments directly or indirectly through a linker. For example, the conjugates can be produced by chemical conjugation to obtain covalent bonds, ionic linkages, or linkages via other chemical interactions such as, but not limited to, van der Waals interactions and hydrophobic interactions. However, the resulting conjugates should be sufficiently stable to allow the molecules to be arranged to remain intact after the binding between the anchors and conjugates.

Conjugation between peptides and PNAs can be achieved using standard techniques used for the synthesis of peptide linkages. See, e.g., Bodanszky, Principles of Peptide Synthesis, 2^{nd} Ed. (1993). These techniques include, but are not limited to, azide coupling; anhydride method using compounds such as carboxycyclic acids derivatives, phosphorous and arsenious acids derivatives, phosphoric acids derivatives, acyloxyphophonium salts, sulfuric acid derivatives, thiol acids, and carbodiimide; and methods using active esters such as active aryl and vinyl esters and reactive hydroxylamine derivatives.

For other molecules, conjugates can be formed using suitable chemical and biological reactions known to those of ordinary skill in the art. For example, molecules that contain reactive groups such as, but not limited to, amino, hydroxyl, sulfhydryl, phenolic, and carboxyl groups can readily provide bonds such as amide, ester, sulfide, disulfide, and thioester bonds when contacted under suitable conditions with other reactive moieties. See generally, Smith, March's Advanced Organic Chemistry: Reactions, Mechanisms, and Structure, 5^{th} Ed. (2001).

Conjugation can be effected by other methods including, but not limited to, alteration in environmental conditions (e.g., temperature, pH and buffer), and/or addition of compounds or molecules that catalyze the formation of a chemical bond (e.g., cross-linking agents). Cross-linking agents can be used to introduce, produce, or utilize reactive groups such as thiols, amines, hydroxyls, and carboxyls, which can then be contacted with other molecules that contain reactive groups to form a bond between the reactive groups. These agents can be used directly or indirectly through a linker to form a conjugate between a molecule to be arranged and a nucleic acid fragment.

Conjugation may be heterofunctional or homofunctional. Examples of heterofunctional conjugation include, but are not limited to: carboxy to amino conjugation using diisopropylcarbodiimide (DIC), disuccinoylcarbonate (DSC), or carbonyldiimidazol (CDI) activators; phosphate-to-amino conjugation using DIC, DSC, or CDI activators; thiol-to-amino conjugation; and aldehyde terminated polymer to aminooxy terminated polymer using methods described in, for example: Tomoko et al., Bioconjugate Chemistry, 14(2): 320-330 (2003); Kisfaludy et al., Ger. Offen., p 74 (1978); www.solulink.com; Kozlov et al., Biopolymers, 73: 621 (2004); Rose, Am. Chem. Soc., 116: 30 (1994); Canne et al., J. Am. Chem. Soc., 117: 2998 (1995); Shao et al., J. Am. Chem. Soc., 117: 3893 (1995); Rodriguez et al., J. Am. Chem. Soc., 119: 9905 (1997); Cervigni et al., Chemistry, Int. Ed. Engl., 35: 1230 (1996); Renaudet et al., Org. Lett., 5: 243 (2003); Forget et al., Chem. Eur. J., 7: 3976 (2001); and “The Universal Linkage System (ULS™) and its use in protein labeling for serum profiling on antibody arrays and antibody immobilization to solid phase,” Kreatech Biotechnology BV, The Netherlands, all of which are incorporated herein by reference.

A particular conjugation is thiol-to-amino conjugation using a heterobifunctional cross-linking agent. Agents that can be used for this purpose include, but are not limited to: 4-succinimidyloxycarbonyl-methyl-α-(2-pyridyldithio)toluene (SMPT); 4-sulfosuccinimidyl-6-methyl-a-(2-pyridyldithio)toluamido-hexanoate (Sulfo-LC-SMPT); N-(k-maleimidoundcanoyloxy)sulfosuccinimide ester (Sulfo-KMUS); succinimidyl-4-(N-maleimidomethyl)cyclohexane-1-carboxy-(6-amidocaproate) (LC-SMCC); N-k-maleimidoundecanoic acid (KMUA); sulfosuccinimidyl-6-[3-(2-pyridyldithio)-propionamido]hexanoate (Sulfo-LC-SPDP); succinimidyl-6-[3-(2-pyridyldithio)-propionamido]hexanoate (LC-SPDP); succinimidyl-4-(p-maleimidophenyl)butyrate (SMPB); sulfosuccinimidyl-4-(p-maleimidophenyl)butyrate (Sulfo-SMPB); succinimidyl-6-(β-maleimidopropionamido)hexanoate (SMPH); sulfosuccinimidyl-4-(N-maleimidomethyl)cyclohexane-1-carboxylate (Sulfo-SMCC); succinimidyl-4-(N-maleimidomethyl)cyclohexane-1-carboxylate (SMCC); N-succinimidyl(4-iodoacetyl)aminobenzoate (SIAB); N-sulfosuccinimidyl(4-iodoacetyl)aminobenzoate (Sulfo-SIAB); N-(g-maleimidobutyryloxy)sulfosuccinimide ester (Sulfo-GMBS); N-(g-maleimidobutyryloxy)succinimide ester (GMBS); m-maleimidobenzoyl-N-hydroxysuccinimide ester (MBS); m-maleimidobenzoyl-N-hydroxysulfosuccinimide ester (Sulfo-MBS); (N-e-maleimidocaproyloxy)sulfosuccinimide ester (Sulfo-EMCS); (N-e-maleimidocaproyloxy)succinimide ester (EMCS); N-e-maleimidocaproic acid (EMCA); N-succinimidyl-(4-vinylsulfonyl)benzoate (SVSB); N-(β-maleimidopropyloxy)succinimide ester (BMPS); N-succinimidyl-3-(2-pyridyldithio)-propionamido (SPDP); succinimidyl-3-(bromoacetamido)propionate (SBAP); N-β-maleimidopropionic acid (BMPA); N-α-maleimidoacetoxy-succinimide ester (AMAS); N-succinimidyl-S-acetyl-thiopropionate (SATP); and N-succinimidyl iodoacetate (SIA). These agents are commercially available, or can be synthesized using methods known in the art.

Examples of homofunctional conjugation include, but are not limited to, thiol-to-thiol conjugation and amino-to-amino conjugation. Agents that can be used to provide thiol-to-thiol conjugate include, but are not limited to: bis-((N-iodoacetyl)piperazinyl)sulfoerhodamine; 1,4-di-[3′-(2′-pyridyldithio)-propionamido]butane (DPDPB); 1,11-bis-maleimidotetraethyleneglycol (BM[PEO]₄); bis-maleimidohexane (BMH); 1,8-bis-maleimidotriethyleneglycol (BM[PEO]₃); 1,6-hexane-bis-vinylsulfone (HBVS); dithio-bis-maleimidoethane (DTME); 1,4-bis-maleimidobutane (BMB); 1,4-bis-maleimidyl-2,3-dihydroxybutane (BMDB); and bis-maleimidoethane (BMOE). These agents are commercially available, or can be synthesized using methods known in the art.

Agents that can be used to provide amino-to-amino conjugate include, but are not limited to: glutaraldehyde; bis(imido esters); bis(succinimidyl esters); diisocyanates; and diacid chlorides. In addition, fixatives such as, but not limited to, formaldehyde and glutaraldehyde may be used to provide amine-amine crosslinking. Other amine-amine conjugation agents include, but are not limited to: ethylene glycol bis(succinimidylsuccinate) (EGS); ethylene glycol bis(sulfosuccinimidylsuccinate) (Sulfo-EGS); bis-[2-(succinimidooxycarbonyloxy)ethyl]sulfone (Sulfo-BSOCOES); bis-[2-(succinimidooxycarbonyloxy)ethyl]sulfone (BSOCOES); dithiobis(succinimidylpropionate) (DPS); 3,3′-dithiobis(sulfosuccinimidylpropionate) (DTSSP); dimethyl 3,3′-dithiobispropionimidate.2HCl (DTBP); disuccinimidyl suberate (DSS); bis(sulfosuccinimidyl) suberate (BS3); dimethyl suberimidate.2HCl (DMS); dimethyl pimelimidate.2HCl (DMP); dimethyl adipimidate.2HCl (DMA); disuccinimidyl glutarate (DSG); methyl N-succinimidyl adipate (MSA); disuccinimidyl tartarate (DST); disulfosuccinimidyl tartarate (Sulfo-DST); and 1,5-flouro-2,4-dinitrobenzene (DFDNB). These agents are commercially available, or can be synthesized using methods known in the art.

5.3 Hybridization

Conjugates between the molecules to be arranged and nucleic acid fragments can be hybridized to anchor structures based on the complementarity between the nucleic acid fragments present in the conjugates and the anchors. Any suitable conditions that would cause a stable binding between two nucleic acids with complementary sequences may be employed for the hybridization. Those conditions are known to those skilled in the art, and can be found, for example, in Current Protocols in Molecular Biology, John Wiley & Sons, N.Y. (1989), 6.3.1-6.3.6, which is incorporated herein by reference.

Hybridization conditions will vary depending upon the nature of the surface-bound nucleic acid and the nature of nucleic acid in the conjugates (Bowtell, Nature Genetics, 21: 25-32 (1999); Brown, Nature Genetics, 21: 33-37 (1999)). Additional hybridization methods and conditions can be found in WO 02/02823 A2 and references cited therein.

Subsequent to hybridization, the anchor-conjugate complexes can be further stabilized using methods known in the art. In one embodiment, the complexes are stabilized using photo-induced crosslinking. Photo-induced crosslinking is well-known in the art, and can be performed using procedures similar to those described, for example, in Hertzberg et al., Applied Microbiology and Biotechnology, 43: 10-17 (1995) and Ansari et al., Proc. Nat'l. Acad. Sci., 99(23): 14706-9 (2002).

5.4 Applications

The arrangements and methods of this invention can be used in a wide variety of applications in numerous fields including, but not limited to, of pharmacology, therapeutics, toxicology, virology and immunology. See, e.g., Protein-Ligand Interactions—From Molecular Recognition to Drug Design, 19: 187-210 and 213-236 (2003).

Exemplary applications include, but are not limited to: establishing binding “fingerprints” for known and unknown proteins; use in “point of care” diagnostics by monitoring up and down regulations of a protein in a cell during a medical treatment; target validation by monitoring the expression of a protein in a cell during the development of therapeutics; and identification of regulation mechanisms of an enzyme.

In some cases, two or more “fingerprints” can be combined to provide an analytical probe for proteins. These probes can be used as “protein chips.” As used herein, the term “chips” refers to certain array formats of molecules of interest. See, e.g., Microarray Biochip Technology, M. Schena Ed. (2000).

The analytical probes can be in two or three dimensional format. Thus, the fingerprints can be arranged on planar and non-planar surfaces. Any surfaces that can be used for immobilization of the anchor structures may be used to build the analytical probes. See supra. In one specific embodiment, the analytical probes, or protein chips, are built as an arrangement of peptides in a capillary tube. In another embodiment, the probes or chips are built in a multi-well plate.

Other applications include, but are not limited to, arrangements of protein-lipid complex molecules, assaying for proteins using single cells immobilized and arranged using receptor-ligand interactions, and monitoring of filtration events using immobilized single cells.

Applications of the invention typically require binding, or association, between the arranged molecules and test molecules. In some embodiments, the binding between conjugate-anchor complexes and the test molecule may be stabilized using methods known in the art. In one embodiment, the binding between conjugate anchor, and/or between the conjugate-anchor complex and the test molecule are stabilized using photo-induced crosslinking. See supra.

Some applications of this invention require the detection of binding between the arranged molecules and test molecules. Any suitable method known in the art for the detection of binding can be used. Examples include, but are not limited to, ELISA, analytical electrophoresis, chemi- and bioluminescence, radioisotopes, staining such as silver staining, fluorescence, and proximity ligation. Description of these analytical methods can be found, for example, in: Sambrook et al., Molecular Cloning, 3^{rd} Ed. (2001); Fredriksson, “Proximity Ligation: Transforming protein analysis into nucleic acid detection through proximity-dependent ligation of DNA sequence tagged protein,” Thesis (2002); and Fredriksson et al., “Protein detection using proximity-dependent DNA ligation assays,” Nature Biotechnology, 20: 473 (2002).

In one embodiment, the binding is detected using chemiluminescence, bioluminescence, silver staining, radioisotopes, or proximity ligation.

5.5 Kits

This invention encompasses kits comprising components used in methods of the invention. The kits may contain one or more of: a multi-well plate, optionally with anchors immobilized on the surface of the wells at addressable locations; anchors comprising nucleic acid fragments; a mixture of conjugates each comprising a nucleic acid fragment and a molecule to be arranged, wherein the nucleic acid fragment has a sequence complementary to at least part of one of the nucleic acid fragments in the anchors; reagents for hybridization, washing, and/or detection.

The conjugates may be included as complexes between nucleic acids and the molecules to be arranged (molecules of interest). Alternatively, the kits can include nucleic acid fragments separately from the molecules to be arranged. In such cases, reagents required for the conjugation of nucleic acids to the molecules can be optionally included in the kits.

As described above, the complexes between the conjugates and anchors, or those between the conjugates, anchors, and test molecules, may be further stabilized. Thus, the kits of the invention may optionally include reagents for further stabilization of complexes formed between the conjugates and anchors, and between the conjugates, anchors, and the test molecules.

In one specific embodiment, this invention encompasses a kit for protein assay comprising:
a multi-well plate, each well containing anchors, each of which is immobilized on the surface of the well and comprises one or more of nucleic acid fragments; and
one or more peptide libraries, each library comprising conjugates, each of which comprising a nucleic acid fragment and a peptide, and wherein the nucleic acid fragment in each conjugate has a sequence complementary to at least part of one of the immobilized nucleic acid fragments in the anchors.

In one embodiment, each of the immobilized nucleic acid fragments is L-DNA or PNA, in particular, L-DNA. In another embodiment, the nucleic acid fragment in each of the conjugate is L-DNA or PNA. In another embodiment, the peptide library can be custom-synthesized according to the specific protein to be assayed.

In addition to the reagents, kits of the invention may contain software or means for viewing, modifying, processing, analyzing, or manipulating the data obtained using methods of this invention. These software or means can be made to perform the functions such as, but not limited to: arraying the images; highlighting a specific locus of interest; moving and zooming in on the loci; removing backgrounds and luminosity from other loci; permitting analysis of the pattern.

Kits can also contain instructions on obtaining the arrangements and further assay protocols. Although not necessarily a part of the kits of this invention, hardware that can perform automated pipetting and analysis are also encompassed by this invention.

### 6. EXAMPLES

6.1 Peptide Arrangement

Tripeptides resulting from all possible combinations of 20 natural amino acids are synthesized (yielding 203=8000 tripeptides) and conjugated to 10-mer PNA fragments.

L-DNA fragments (30-mers), in which each 10-mer unit is complementary to at least one of the PNA fragments used for the conjugates, are spotted on the bottom of a well in a 96 well plate. Using an equipment with a resolution of 100 micrometer center-to-center (e.g., contact printing: Genetix (http://www.genetix.com/MicroarrayNews/Page1.htm); Genomic Solutions (GeneMachine Accent OmniGrid, BioForce Nanosciences), or non-contact printing: acoustic wave deposition (LabCyte, EDC Biosystem); or Phalanx (Taiwan, www.phalanxbiotech.com/english/technology-temp.htm#TechNotes)), 1600 different L-DNA fragments are immobilized in each of the wells. L-DNAs are immobilized using standard chemical conjugation (e.g., using conjugation reagents from EDC Biosystems), optionally with photo-activation using procedures substantially similar to those described in U.S. Pat. No. 6,033,784. Alternatively, L-DNAs may be immobilized using oxime coupling.

PNA-tripeptide conjugates are added to each of the wells and the mixture is incubated to allow the PNA-tripeptide conjugates to hybridize to the immobilized L-DNAs. The plate is washed to remove excess conjugates. After hybridization and washing, a 96 well plate which contains 153,600 (1600×96) different peptide arrangements is generated.

The number of arrangements can be varied (e.g., increased) by allowing the reverse orientation arrangement of tri-peptides, or by using the alpha anomeric version to generate the sequence motif. Additional arrangements can be obtained by placing spacers in between the complementary L-DNA motifs in the stem, thereby changing the distance of the peptide conjugates. This can also generate “3D protein binding pockets” with modified pocket sizes. In addition, using mathematical models and several rounds of optimization to define the number of L-DNA templates, the spacers, the number of PNA-peptide-conjugates, the number of compartments (wells), and the pipetting steps, a large number of protein binding pockets can be generated from a very small library of PNA-peptide-conjugates. See, e.g., Green et al., Mini-Reviews in Medicinal Chemistry, 4(10): 1067-1076 (2004) and Konno, Kagaku to Kogyo, 56(10): 1151 (2003).

6.2 Binding Fingerprints of MHC Complex

A “fingerprint” of an individuals immune system can be generated using the library of protein binding pockets obtained using methods of this invention. A data base of human MHC fingerprints can then be generated, allowing convenient identification of, for example, potential donors for bone marrow or organ transplantation. For detailed discussion of human MHC complex, see, e.g., Rammensee, Nature, 419: 443 (2002).

A library of protein binding pockets in a 96 well plate is prepared according to the methods described in Section 6.1, above. Proteins from MHC complexes are added to the well and allowed to bind to the binding pockets. The well is washed to remove unbound and/or excess proteins.

Commercially available MHC class I and II antibodies (tethered to AP) are added and allowed to bind to the MHC proteins bound to the pockets. Binding is detected using the light signal generated by degradation of a dioxetane substrate. The pattern of binding is recorded as an image or a data set.

6.3 Combinatorial Hybridization to Mimic G-Protein Coupled Receptors

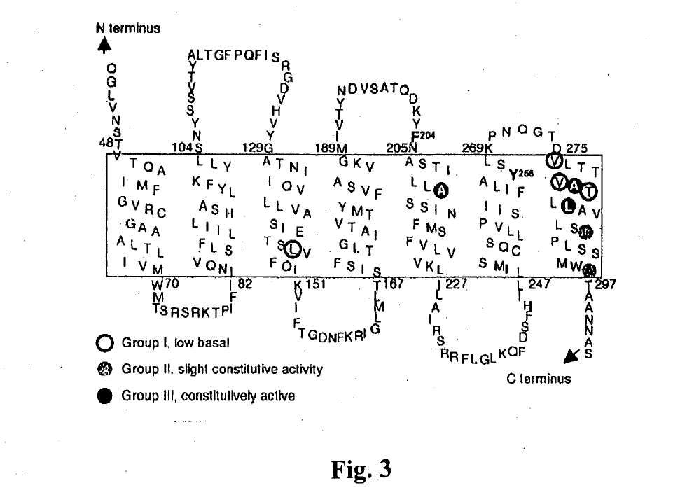

G-Protein Coupled Receptors (“GPCRs”) are a family of proteins that transduce certain extra-cellular signals to the interior of the cells. Their involvement in the growth and progression of androgen independent prostate cancer cells have been implicated. For detailed discussion, see, e.g., Raj et al., J. Urol., 167(3): 1458-1463 (2002). An exemplary GPCR Ste2p has the structure shown in FIG. 3.

Using the combinatorial hybridization methods described in Section 6.1, above, arrangements that resemble the extra- and intra-cellular loops of Ste2p are generated. By hybridizing conjugates comprising candidate molecules of potential interaction partners of Ste2p to the arrangements, interactions occurring on the cell surface, and processes and specificity of such interactions, in connection with the cell signaling, can be studied.

All of the references cited herein are incorporated by reference in their entireties.

While the invention has been described with respect to the particular embodiments, it will be apparent to those skilled in the art that various changes and modifications may be made without departing from the spirit and scope of the invention as recited by the appended claims.

What is claimed is:

1. An array comprising a plurality of conjugates and a plurality of anchors, wherein:
each of the conjugates comprises a molecule bound to a nucleic acid fragment, wherein the molecule of each conjugate is not the same as the molecule of any of the other conjugates;
each of the anchors is immobilized on a surface and comprises at least two nucleic acid fragments; and
the nucleic acid fragment of each conjugate is hybridized to at least one of the nucleic acid fragments of the anchors.

2. The array of claim 1, wherein the molecules are peptides, peptoids, proteins, steroids or analogues thereof, hormones, carbohydrates, polycarbohydrates, aminoglycosides, aptamers of L or D oligoribonucleotides, nucleoside antibiotics, L-nucleoside analogues, oligoglycosids, macrolids, polyenes, oligolactones, polyethers, tetracycline, anthracycline, p-chinoid macrolactams, terpenoids, isopren and analogues thereof, peptide antibiotics, or benzodiazepine.

3. The array of claim 2, wherein the peptides are tri-peptides.

4. The array of claim 1, wherein at least two of the anchors comprise the same nucleic acid fragment.

5. The array of claim 1, wherein the nucleic acid fragment in each conjugate is L-DNA or PNA.

6. The array of claim 1, wherein at least one of the nucleic acid fragments in each anchor is L-DNA or PNA.

7. The array of claim 6, wherein at least one of the nucleic acid fragments in each anchor is L-DNA.

8. A method of arranging molecules comprising:
(a) immobilizing a first set of nucleic acid fragments with known sequences in a predetermined pattern on a surface to form anchors;
(b) contacting the anchors with a mixture comprising conjugates of a second set of nucleic acid fragments and the molecules, wherein the nucleic acid fragment in each conjugate has a sequence complementary to at least part of one of the nucleic acid fragments in the anchors; and
(c) incubating the anchors and the mixture for a time and under conditions sufficient for the conjugates to bind to the anchors, thereby arranging the molecules.

9. The method of claim 8, wherein the molecules to be arranged are peptides, peptoids, proteins, steroids or analogues thereof, hormones, carbohydrates, polycarbohydrates, aminoglycosides, aptamers of L or D oligoribonucleotides, nucleoside antibiotics, L-nucleoside analogues, oligoglycosids, macrolids, polyenes, oligolactones, polyethers, tetracycline, anthracycline, p-chinoid macrolactams, terpenoids, isopren and analogues thereof, peptide antibiotics, or benzodiazepine.

10. The method of claim 9, wherein the peptides are tri-peptides.

11. The method of claim 9, wherein the peptides comprise peptoids or D-amino acids.

12. The method of claim 8, wherein each of the anchors comprises two or more nucleic acid fragments, each of which is capable of binding at least one of the conjugates.

13. The method of claim 12, wherein at least two of the anchors comprise the same nucleic acid fragment.

14. The method of claim 8, wherein the surface is a porous surface.

15. The method of claim 8, wherein the surface is a well of a multi-well plate.

16. The method of claim 8, wherein at least one of the nucleic acid fragments in each anchor is L-DNA or PNA.

17. The method of claim 16, wherein at least one of the nucleic acid fragments in each anchor is L-DNA.

18. The method of claim 8, wherein the immobilization is achieved using chemical conjugation or oxime coupling.

19. The method of claim 8, which further comprises stabilizing the anchor/conjugate complexes using photo-induced crosslinking.

20. The method of claim 8, wherein the nucleic acid fragment in each conjugate is L-DNA or PNA.

21. A method of characterizing a protein comprising:
(a) immobilizing a first set of nucleic acid fragments with known sequences in a predetermined pattern on a surface to form anchors;
(b) contacting the anchors with a mixture comprising conjugates of a second set of nucleic acid fragments and peptide fragments, wherein the nucleic acid fragment in each conjugate has a sequence complementary to at least part of one of the nucleic acid fragments in the anchors;
(c) incubating the anchors and the mixture for a time and under conditions sufficient for the conjugates to bind to the anchors to provide an array of anchor-conjugate complexes;
(d) contacting the array with the protein for a time and under conditions sufficient for the protein to bind to one or more of the complexes; and
(e) detecting the binding of the protein to the complexes to obtain a binding pattern;
wherein the binding pattern is characteristic of the protein.

22. The method of claim 21, wherein the characteristics of two or more of proteins are combined to provide an analytical probe for proteins.

23. The method of claim 22, wherein the analytical probe is a protein chip.

24. The method of claim 22, wherein the analytical probe is an arrangement of peptide sequences in a capillary tube.

25. The method of claim 21, wherein the protein is a Major Histo-Compatibility (MHC) complex.

26. The method of claim 21, wherein the binding is detected by chemiluminescence, bioluminescence, silver staining, radioisotopes, or proximity ligation.

27. The method of claim 21, wherein each of the anchors comprises two or more nucleic acid fragments, each of which is capable of binding at least one of the conjugates.

28. The method of claim 27, wherein at least two of the anchors comprise the same nucleic acid fragment.

29. The method of claim 21, wherein the peptide fragments are tri-peptides.

30. The method of claim 21, wherein the surface is a porous surface.

31. The method of claim 21, wherein the surface is a well on a multi-well plate.

32. The method of claim 21, wherein at least one of the nucleic acid fragments in each of the anchors is L-DNA or PNA.

33. The method of claim 32, wherein at least one of the nucleic acid fragments in each of the anchors is L-DNA.

34. The method of claim 21, wherein the immobilization is achieved using chemical conjugation or oxime coupling.

35. The method of claim 21, which further comprises stabilizing the anchor/conjugate complexes using photo-induced crosslinking.

36. The method of claim 21, wherein the method further comprises stabilizing the anchor/conjugate/protein complexes using photo-induced crosslinking.

37. The method of claim 35, wherein the method further comprises stabilizing the anchor/conjugate/protein complexes using photo-induced crosslinking.

38. The method of claim 21, wherein the nucleic acid fragment in the conjugate is L-DNA or PNA.

39. A kit for a protein assay comprising:
a multi-well plate, each well containing anchors, each of which is immobilized on the surface of the well and comprises one or more of nucleic acid fragments; and
one or more peptide libraries, each library comprising conjugates, each of which comprising a nucleic acid fragment and a peptide, and wherein the nucleic acid fragment in each conjugate has a sequence complementary to at least part of one of the immobilized nucleic acid fragments in the anchors.

40. The kit of claim 39, wherein each of the immobilized nucleic acid fragments is L-DNA or PNA.

41. The kit of claim 40, wherein each of the immobilized nucleic acid fragments is L-DNA.

42. The kit of claim 41, wherein the nucleic acid fragment in each of the conjugate is L-DNA or PNA.
